(19) United States
(12) Patent Application Publication
(10) Pub. No.: US 2014/0142124 A1
(43) Pub. Date: May 22, 2014

(54) Compositions for Topical Application Having Androgenic Actions

(71) Applicant: Aventis Pharma S.A., Antony (DE)

(72) Inventors: Karl Theodor Kraemer, Langen (DE); Manfred Bohn, Hofheim (DE)

(21) Appl. No.: 13/916,488

(22) Filed: Jun. 12, 2013

Related U.S. Application Data
(63) Continuation of application No. 13/436,545, filed on Mar. 30, 2012, now abandoned.
Application No. 13/436,545 is a continuation of application No. 12/292,521, filed on Nov. 20, 2008, now abandoned.
Application No. 12/292,521 is a division of application No. 09/425,742, filed on Oct. 22, 1999, now abandoned.

(30) Foreign Application Priority Data
Oct. 23, 1998 (DE) 19848856.4
Jan. 12, 1999 (DE) 19900749.7

(51) Int. Cl. A61K 31/421; A61K 31/4166; A61K 45/06; C07D 263/06; C07D 233/70

(52) U.S. Cl. CPC A61K 31/421; C07D 263/06; C07D 233/70; A61K 45/06; A61K 31/4166
USPC 514/263.36; 514/374; 514/392

(57) ABSTRACT

A composition comprising at least one physiologically tolerated film-forming agent, at least one physiologically tolerated solvent, at least one plasticizer and a compound of the formula I

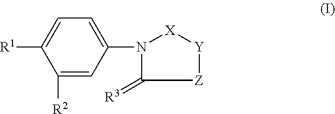

or a stereoisomeric form or a physiologically tolerated salt of any of the foregoing. The composition is suitable for treatment of androgenic alopecia or hirsutism, that is, for avoiding undesirable hair growth, and for treatment of seborrhea and acne, and can furthermore be employed in cosmetics.

Androgenic alopecia is the most frequent form of hair loss, which can occur both in men and in women. The term “androgenic alopecia” is understood as meaning hair deficiency states the cause of which is a genetically determined hypersensitivity of the hair root to 5α-dihydrotestosterone.

A typical example of androgenic alopecia is the common baldness in men, that is, male pattern baldness. However, androgenic alopecia can also occur in women of sexually mature age—with or without the clinical features of male baldness.

A prerequisite of treatment of androgenic hair loss is early interruption of the pathogenetic processes which cause degeneration of the hair follicle. To achieve a normalization of the hair cycle, that is, prolonging of the growth phase of the hair, it is necessary to reduce the biologically active amount of androgen at the follicle. When endocrinopathies have been ruled out and medicaments which comprise testosterone or other substances having an androgenic action have been discontinued, inhibition of androgen stimulation at the target organ is necessary. To achieve this aim, two routes are theoretically conceivable: firstly, inhibition of the activity of the 5α-reductase and therefore a reduction in the conversion of testosterone into 5α-dihydrotestosterone, for example by estrogen, and secondly, blocking of the dihydrotestosterone-sensitive receptor protein, for example by antiandrogens.

Since all systemic treatment measures for androgenic alopecia are directed against the androgen action, they can be used on women of child-bearing age only with simultaneous contraception. After introduction of oral contraceptives, it was found that the course of androgenic alopecia and its concomitant symptoms is influenced favorably or unfavorably depending on whether an estrogen-emphasized preparation or a preparation with a residual androgenic action is administered.

In the absence of another risk-free alternative with a more potent action, estrogen-containing hair lotions have hitherto been described for treatment of androgenic alopecia in men. In women, this local treatment is recommended as an assisting measure, and the main emphasis is placed on systemic treatment.

All patients are instructed to treat the region of the scalp still covered with hair and not the areas which are already bald. In many cases, it is possible to alleviate or to stop the episodes of hair loss with the aid of these local measures.

Antiandrogens having a topical action are known from French Patent 2,693,461 and U.S. Pat. No. 5,411,981 (4-[3-(4-hydroxybutyl)-4,4-dimethyl-2,5-dioxo-1-imidazolidinyl]-2-(trifluoromethyl)benzonitriles) and from PCT Application WO 98/05654 (3-aryl-2,4-dioxo-oxazolidines), but are currently not yet generally commercially available for treatment purposes.

Both classes of substance show a high bonding affinity for the androgen receptor at the hair root after topical application, with virtually no systemic activity.

Due to the teratogenicity of antiandrogens, intrinsic to the substances, with an influence on sex differentiation in the late stage of pregnancy, the substances mentioned cannot be used in the form of conventional aqueous/alcoholic hair lotions because of the occurrence of precipitates of the substances at the application site after evaporation of the solvent and the associated toxicological risk of transfer of the substance to pregnant women. Furthermore, delayed release of the active compounds over a relatively long period of time, in order to avoid high systemic concentrations of the active substance and the associated occurrence of systemic antiandrogenic effects, is not guaranteed by conventional formulations for application to the scalp.

In order to make the antiandrogenic active compounds in the above-mentioned references available for a reliable and effective treatment, it was therefore necessary to discover compositions which do not have the disadvantages described for conventional scalp treatment compositions.

The object is achieved by the compositions according to the invention, comprising one or more topical antiandrogens according to U.S. Pat. No. 5,411,981 or WO 98/05654, the disclosures of both of which are explicitly incorporated herein by reference, a physiologically tolerated volatile solvent or solvent mixture, a plasticizer and one or more physiologically acceptable film-forming agents which, after drying of the composition, form flexible films which adhere to the scalp and are capable of releasing the active compounds employed in a controlled manner and over a certain period of time. Moreover, the undesirable precipitation of the active compound at the application site is prevented by the compositions according to the invention.

The invention, therefore relates to a composition comprising at least one physiologically tolerated film-forming agent, at least one physiologically tolerated solvent, at least one plasticizer and a compound of the formula I

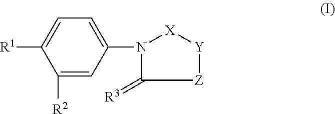

or a stereoisomeric form or a physiologically tolerated salt of any of the foregoing, in which:
R¹ is 1) —CN,
   2) —NO₂,
   3) a halogen, or
   4) (C₁-C₄)-alkyl-C(O)—OH;
R² is 1) —CF₃.
   2) a halogen, or
   3) —CN;
R³ is 1) ═O,
   2) ═S, or
   3) ═NH;
X is 1) a radical of formula II

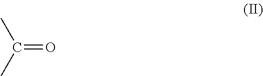

or
2) a radical of formula III

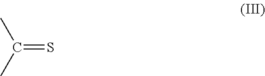

or X and Y together form a group of formula IV

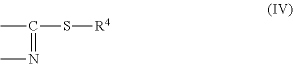

in which R⁴ is 1) hydrogen atom,
   2) (C₁-C₆)-alkyl-,
   3) (C₂-C₆)-alkenyl-, or
   4) (C₁-C₆)-alkyl-,
   wherein the alkyl is mono- to trisubstituted by
      4.1 —OH,
      4.2 halogens,
      4.3 —O—(C₁-C₄)-alkyl,
      4.4 —CN, or
      4.5 —SH;
Y is 1) a radical of formula V

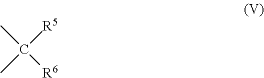

in which:
R⁵ is, independently of R⁶, a hydrogen atom or (C₁-C₄)-alkyl, wherein the alkyl is unsubstituted or mono- to tetrasubstituted by halogens, and R⁶ is, independently of R⁵, (C₁-C₄)-alkyl, wherein the alkyl is unsubstituted or mono- to trisubstituted, by
   a) halogens,
   b) phenyl-(CH₂)ₘ—, wherein the phenyl is unsubstituted or mono- to trisubstituted, independently of one another, by —COOH, —CN, or —CF₃, and m is the integer zero, 1, 2, 3, 4, 5, or 6,
   c) —COOH,
   d) —CN, or
   e) —CF₃, or
2) a radical of formula VI,

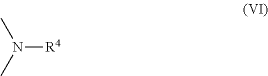

in which R⁴ is as defined above; and
Z is 1) —O— or
   2) a radical of formula VII

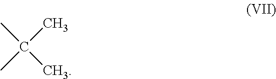

A preferred composition is that comprising a compound of the formula I in which
R¹ is 1) —CN,
   2) —NO₂, or
   3) a halogen;
R² is 1) —CF₃ or
   2) a halogen;
R³ is 1) ═O or
   2) ═S;
X is the radical of formula II or III, or
X and Y together form the group of formula IV,
   in which R⁴ is as defined in claim 1;
Y is the radical of formula VI,
   in which R⁴ is as defined in claim 1; and
   Z is the radical of formula VII.

A composition which is currently preferred is that comprising a compound of the formula I in which
R¹ is —CN;
R² is —CF₃;
R³ is =0;
X its the radical of formula II;
Y is the radical of formula VI, in which R⁴ is hydrogen; and
Z is —O— or the radical of formula VII.

Compounds of the formula I such, as 4-[3-(4-hydroxybutyl)-4,4-dimethyl-2,5-dioxo-1-imidazolidinyl]-2-(trifluoromethyl)benzonitrile or 4-(5-methyl-2,4-dioxo-5-trifluoromethyl)-oxazolidin-3-yl)-2-(trifluoromethyl)benzonitrile are mentioned as currently believed to have particular promise.

For the present invention, the term “halogen” is understood as meaning fluorine, chlorine, bromine or iodine. The term “alkyl” or “alkenyl” is understood as meaning hydrocarbon radicals in which the carbon chains are straight-chain or branched. The alkenyl radicals can furthermore also contain several double bonds. The term “physiologically tolerated solvent” is understood as meaning, for example, water or (C₁-C₆)-alcohols, such as methanol, ethanol, propanol, isopropanol, butanol, pentanol, or hexanol. However, mixtures of the solvents can also be employed. The term independent, when used to describe the relationship of radicals, atoms, substituents, functional groups, etc., means that each of the radicals, atoms, substituents, functional groups, etc. may be the same or different from the other, or some radicals, atoms, substituents, functional groups, etc., may be the same while the others may be different. The term “derivative” means, when describing compound, a compound produced or obtained from another and containing the elements of the parent substance. The adverbs and adjectives of each term are readily apparent to those skilled in the art.

Plasticizers are substances which impart to brittle compositions, for example film-forming substances, suppleness, and flexibility. The release profile of substances from films can moreover also be controlled by the nature and amount of the plasticizer added. Various classes of substances are possible suitable plasticizers, in particular ethoxylated compounds, panthenol and esters of adipic or sebacic acid. Preferably, plasticizers are chosen from potyoxyethylated castor oil, ethoxylated cholesterol, and panthenol.

Film-forming agents are substances of varying composition which have the feature that, when dissolved in water or other suitable solvents, they form films on the skin after the water or the solvent has evaporated, these films being capable, inter alia, of releasing incorporated active compounds in a controlled manner over a certain period of time.

In contrast to thickeners, which are added to liquid compositions to establish a certain viscosity, film-forming agents influence the viscosity of a liquid to only a small extent. A disadvantage of thickeners is the poor dispersibility of the application form.

The compositions according to the invention are primarily distinguished by a uniform release, proceeding over a certain period of time, of the compound of the formula I from the elastic film which forms after application of the composition and adheres firmly to the skin. This ensures that therapeutically active antiandrogen concentrations are achieved at the target organ—the hair root—over a relatively long period of time, without high blood level concentrations occurring in the short term, which of course lead to a systemic stress on the patient.

The compositions are preferably in the form of a liquid composition, such as hair lotions or hair tonics, which can comprise as the main constituents water, and also aqueous (C₁-C₆)-alcohol, such as, for example, ethanol, propanol, or isopropanol; and furthermore lotion and semi-solid compositions, such as emulsions, creams, gels or ointments. If appropriate, the compositions can also be in the form of aerosols.

Suitable film-forming agents are, for example, naturally occurring substances, such as alginic acid, alginates, collagen, collagen derivatives, hydrolyzed wheat proteins, carrageenan, cellulose, cellulose derivatives, chitosan, chitosan derivatives, keratin hydrolysates, protein hydrolysates, gelatin, guar gum, guar gum derivatives, hydrolyzed elastin, hydrolyzed milk proteins, hydrolyzed silk proteins, hydrolyzed soya protein, hydrolyzed oat proteins, copolymer of hydroxyethylcellulose and dimethyldiallylammonium chloride, hyaluronic acid, hyaluronates, tragacanth, and xanthan; and synthetic substances, such as acrylate/acrytamide copolymers, acrylate copolymers, acrylate/octylacrylamide copolymers, acrylic acid ester copolymers, methacrylic acid copolymers, adipic acid/dimethyl-aminohydroxypropyldiethylenetriamine copolymers, methacrylic acid/methacrylic acid ester copolymers neutralized with 2-amino-2-methylpropanol, polyacrylic acid crosslinked with pentaerythritol ethers or sugar allyl ethers, polysiloxane/polyalkyl polyether copolymers, polysiloxanes, ethylene/acrylic acid ester copolymers, ethylene/vinyl acetate copolymers, methacryloylethylbetaine/methacrylic acid copolymers, octylacrylamide/acrylic acid ester/butylaminoethylmeth-acrylic-acid copolymers, quaternized polyvinylpyrrolidone-dimethylaminoethylmethacrylic acid esters, polyvinylpyrrolidone/imidazolinium methochloride copolymers, sodium acrylate/dimethyldiallylammonium chloride copolymers, dimethyldiallylammonium chloride/sodium acrylate/acrylamide terpolymer, poly(dimethylsiloxane-copolyol-phosphopanthenoate), poly(methyl vinyl ether-maleic anhydride), poly(methyl vinyl ether-maleic acid monoalkyl ester), poly(vinylpyrrolidone), terpolymers based on pyrrolidone and acrylic acid compounds, poly(vinylpyrrolidone-dimethylaminoethylmethacrylic acid), polyvinylpyrrolidone/eicosene copolymer, polyvinylpyrrolidone/methacrylic acid ester/methacrylic acid terpolymer, polyvinylpyrrolidone/hexadecene copolymer, polyvinyl-pyrrolidone/polycarbamyl polyglycol ester, polyvinylpyrrolidone/vinyl acetate copolymer, vinylimidazolium methochloride/vinylpyrrolidone copolymer, acrylic acid/acrylic acid ester copolymers and terpolymer of vinylpyrrolidone, vinyl acetate, and vinyl propionate.

As additives, the compositions according to the invention can also comprise at least one circulation-promoting compound, such as dihydralazine, diisopropylamine or diazoxide, or calcium antagonists, such as nifedipine, nicardipine, verapamil, diltiazem, nisoldipine, nitrendipine, nivaldipine, isradipine, felodipine, nimodipine, gallopamil, fendiline, flunarizine, amlodipine, diperdipine, fluspirilene, primozide, fantofarone, nicergoline or cyclandelate, 6-amino-4-piperidino-1,2-dihydro-1-hydroxy-2-iminopyrimidine (minoxidil), angiotensin converting enzyme inhibitors, such as quinapril, lisinopril, benzazepril, captopril, ramipril, fosinopril, cifazapril or trandoldpril, methylxanthine compounds, such as pentoxifyllin, propentofyllin or torbafyllin, or a mixture thereof.

Suitable additives are also at least one sodium channel opener, such as 1-cyano-2-(1,1-dimethyl-propyl)-3-(3-pyridyl)guanidine, or 5-alpha-reductase inhibitors, such as N-tert-butyl-3-oxo-4-aza-5α-androst-1-ene-17β-carboxamide. Other suitable additives are also at least one hair growth-promoting compound, such as an inner salt of 2,4-diamino-6-alkoxy-3-sulfoxypyrimidine hydroxide having 1 to 6 carbon atoms in the alkoxy radical, as described in EP 0 427 625; for example, the inner salt of 2,4-diamino-6-butoxy-3-sulfoxypyrimidine hydroxide, or pyridine 1-oxide derivatives as described in WO 92 21317, for example 2,6-diamino-4-piperidinopyridine, or 2,6-diamino-1,3,5-triazine derivatives as described in WO 91 19701, far example 2,6-diamino-4-butoxy-1,3,5-triazine 1-oxide. Mixtures of the additives mentioned are also suitable.

The compositions according to the invention can comprise as further additives the hair- and scalp-care substances customary in cosmetics and medical active compounds, such as, for example, antidandruff agents, preparations having an antiseborrheic action, substances having a keratolytic and keratoplastic action, such as salicylic acid, allantoin, sulfur preparations, urea and ceramides, antimicrobial agents, vitamins, plant or organ extracts, hormones, corticoids, hyperemic agents, such as nicotinic add and derivatives thereof, organic acids, such as citric acid, orotic acid, liponic acid and amino acids, polyethoxylated fatty alcohols, fatty acids, sorbitan fatty acid esters, alkyl phosphates and oils, for example fatty acid esters, and furthermore preservatives, dyestuffs and perfume oils. It is currently believed that the additives should be compatible with antiandrogenic substances such that the additives do not inhibit the hair growth action thereof.

The treatment of androgenic alopecia can be carried out reliably and effectively with the compositions according to the invention. This is an extremely important finding, in view of the poor treatment results to date.

The compositions according to the invention are also suitable for treatment of hirsutism, that is, for avoiding undesirable hair growth, and for treatment of seborrhea and acne.

The compositions according to the invention in general comprise the active compound in an amount of 0.01 percent by weight to 10 percent by weight, preferably 0.1 to 5 percent by weight.

In liquid compositions, the amount of solvents is from 85 percent by weight to 97.5 percent by weight and the amount of plasticizer is from 0.05 percent by weight to 2.5 percent by weight. Semi-solid compositions comprise 50 percent by weight to 75 percent by weight of solvent and the amount of plasticizer is from 0.05 percent by weight to 2.5 percent by weight.

The invention furthermore relates to the use of the compositions according to the invention in cosmetics.

The compositions according to the invention are in general prepared in a manner known per se by dissolving the substances having an antiandrogenic action in the particular vehicle in question.

The composition according to the invention has, for example, the following composition:

### EXAMPLE 1

| | |
|---|---|
| 4-[3-(4-Hydroxybutyl)-4,4-dimethyl-2,5-dioxo-1- | 5.0% |
| imidazolidinyl]-2-(trifluoromethyl)benzonitrile | |
| Vinylimidazolium methochloride/vinylpyrrolidone | 2.5% |
| copolymer | |
| (Luviquart ® FC 550) | |
| Polyethoxylated hydrogenated castor oil | 2.5% |
| (Cremophor ® RH 410) | |
| Ethanol 96% | 63.0% |
| Demineralized water | 27.0% |

The percentage amounts stated are based on the weight. The composition is prepared by dissolving the various components in water.

### EXAMPLE 2

| | |
|---|---|
| 4-[3-(4-Hydroxybutyl)-4,4-dimethyl-2,5-dioxo-1- | 1.0% |
| imidazolidinyl]-2-(trifluoromethyl)benzonitrile | |
| Ethoxylated cholesterol | 1.0% |
| (Solulan ® C-24) | |
| Polyvinylpyrrolidone K 30 | 2.0% |
| Partly hydrolyzed collagen | 1.5% |
| (Lanasan CL ®) | |
| Ethyl alcohol 96% | 20.0% |
| Preservative | |
| Demineralized water | 74.5% |

### EXAMPLE 3

| | |
|---|---|
| 4-[3-(4-Hydroxybutyl)-4,4-dimethyl-2,5-dioxo-1- | 0.5% |
| imidazolidinyl]-2-(trifluoromethyl)benzonitrile | |
| Ethyl alcohol | 25.0% |
| Methyl vinyl ether/maleic acid butyl ester copolymer | 1.5% |
| (Gantrez ® ES-425) | |
| Tris(hydroxymethyl)aminomethane | 0.03% |
| Panthenol | 0.5% |
| Demineralized water | 72.47% |

### EXAMPLE 4

| | |
|---|---|
| 4-[3-(4-Hydroxybutyl)-4,4-dimethyl-2,5-dioxo-1- | 2.0% |
| imidazolidinyl]-2-(trifluoromethyl)benzonitrile | |
| Vinylimidazolium methochloride/vinylpyrrolidone | 2.0% |
| copolymer (Luviquart ® FC 550) | |
| Polyethoxylated hydrogenated castor oil | 2.0% |
| (Cremophor ® RH 410) | |
| Ethanol 96% | 40.0% |
| Demineralized water | 54.0% |

### EXAMPLE 5

| | |
|---|---|
| 4-(5-Methyl-2,4-dioxo-5-trifluoromethyl)oxazolidin-3- | 2.0% |
| yl)-2-trifluoromethylbenzonitrile | |
| Vinylimidazolium methochloride/vinylpyrrolidone | 2.0% |
| copolymer | |
| (Luviquart ® FC 550) | |
| Polyethoxylated hydrogenated castor oil | 2.0% |
| (Cremophor ® RH 410) | |
| Ethanol 96% | 40.0% |
| Demineralized water | 54.0% |

The delayed release of the active compound from the compositions according to the invention is demonstrated in permeation tests on human skin covered with hair and without hair cover. The measurement method used enables the release of an active compound from a particular composition and the subsequent permeation through human skin to be tested.

As a control example,

| | |
|---|---|
| 4-[3-(4-hydroxybutyl)-4,4-dimethyl-2,5-dioxo-1- | 5.0% |
| imidazolidinyl]-2-(trifluoromethyl)benzonitrile | |
| is dissolved | |
| in ethanol 96% | 66.5% |
| and demineralized water | 28.5%. |

Permeation Test on Skin Covered with Hair and Without Hair Cover

The permeation of the active compound is measured by means of the time-resolved ATR technique (time-resolved infrared attenuated total reflection, see Th. M. Bayerl et al.; J. Invest. Dermatol. 105:291-295, 1995):

100 μL of the test composition (control example) are applied to a defined area of the upper side of the human skin, covered with hair and without hair cover, lying on the measurement crystal. The permeation of the active compound can be observed with the aid of the IR band at 1323 cm⁻¹ characteristic of 4-[3-(4-hydroxybutyl)-4,4-dimethyl-2,5-dioxo-1-imidazolidinyl]-2-(trifluoromethyl)benzonitrile.

It was found here that about 90% of the amount of active compound applied permeates within 24 hours both through the skin covered with hair and through the skin without hair cover.

However, there were differences in the rate of permeation between the two pieces of skin. While the amount of active compound which has permeated already asymptotically approaches the end value after about 7 hours when skin covered with hair is used, the substance permeates virtually uniformly through skin without hair cover over 24 hours.

After application of a composition according to the invention, for example according to Example 1, to skin containing hair follicles—such as exists with androgenic alopecia—a uniform permeation of the active compound over 24 hours, as after application of the control composition to skin without hair cover, was likewise achieved.

Furthermore, when the composition according to the invention was used, no precipitation of the active compound at the application site occurred after the solvent had evaporated, in contrast to the control composition.

The present invention may be embodied in other specific forms without departing from its spirit or essential characteristics. The described embodiments are to be considered in all respects as illustrative only and not restrictive. The scope of the invention is, therefore, indicated by the appended claims rather than by the foregoing description. All changes which come within the meaning and range of equivalency of the claims are to be embraced within their scope.

What is claimed is:

1. A composition comprising:
a) at least one physiologically tolerated film-forming agent;
b) at least one physiologically tolerated solvent;
c) at least one plasticizer, and
d) a compound of the formula I 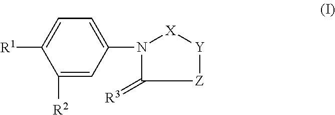
or a stereoisomeric form or a physiologically tolerated salt of any of the foregoing, in which:
R¹ is 1) —CN,
2) —NO₂,
3) a halogen, or
4) (C₁-C₄)-alkyl-C(O)—OH;
R² is 1) —CF₃,
2) a halogen, or
3) —CN;
R³ is 1) ═O,
2) ═S, or
3) ═NH;
—X is 1) a radical of formula II 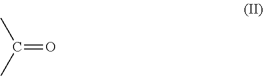
or
2) a radical of formula III 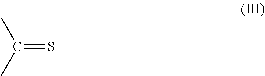
or X and Y together form a group of formula IV 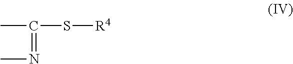
in which R⁴ is 1) hydrogen atom,
2) (C₁-C₆)-alkyl-,
3) (C₂-C₆)-alkenyl-, or
4) (C₁-C₆)-alkyl-,
wherein the alkyl is mono- to trisubstituted by
4.1 —OH,
4.2 halogens,
4.3 —O—(C₁-C₄)-alkyl,
4.4 —CN, or
4.5 —SH;
Y is 1) a radical of formula V 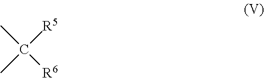
in which:
R⁵ is, independently of R⁶, a hydrogen atom or (C₁-C₄)-alkyl, wherein the alkyl is unsubstituted or mono- to tetrasubstituted by halogens, and
R⁶ is, independently of R⁵, (C₁-C₄)-alkyl, wherein the alkyl is unsubstituted or mono- to trisubstituted, by
a) halogens,
b) phenyl-(CH₂)ₘ—, wherein the phenyl is unsubstituted or mono- to trisubstituted, independently of one another, by —COOH, —CN, or —CF₃, and m is the integer zero, 1, 2, 3, 4, 5, or 6,
c) —COOH,
d) —CN, or
e) —CF₃, or
2) a radical of formula VI, 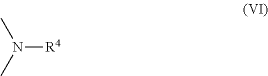
in which R⁴ is as defined above; and
Z is 1) —O— or
2) a radical of formula VII

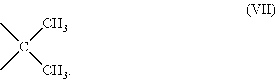

2. A composition as claimed in claim 1, wherein the compound of formula I is a compound in which:
R¹ is 1) —CN,
2) —NO₂ or
3) a halogen;
R² is 1) —CF₃ or
2) a halogen;
R³ is 1) ═0 or
2) ═S;
X is the radical of formula II or III, or
X and Y together form the group of formula IV,
in which R⁴ is as defined in claim 1;
Y is the radical of formula VI,
in which R⁴ is as defined in claim 1; and
Z is the radical of formula VII.

3. A composition as claimed in claim 1, wherein the compound of formula I is a compound in which:
R¹ is —CN;
R² is —CF₃;
R³ is =0;
X is the radical of formula II;
Y is the radical of formula VI, in which R⁴ is hydrogen; and
Z is —O— or the radical of formula VII.

4. A composition as claimed in claim 1, wherein the compound of formula I is chosen from 4-[3-(4-hydroxybutyl)-4,4-dimethyl-2,5-dioxo-1-imidazolidinyl]-2-(trifluoromethyl)benzonitrile and 4-(5-methyl-2,4-dioxo-5-trifluoromethyl)-oxazolidin-3-yl)-2-(trifluoromethyl)-benzonitrile.

5. A composition as claimed in claim 1, wherein the at least one plasticizer is chosen from ethoxylated compounds, panthenol, esters of adipic acid, and esters of sebacic acid.

6. A composition as claimed in claim 5, wherein the at least one plasticizer is chosen from polyoxyethylated castor oil, ethoxylated cholesterol, and panthenol.

7. A composition as claimed in claim 1, wherein the at least one physiologically tolerated solvent is chosen from water and (C₁-C₆)-alcohols.

8. A composition as claimed in claim 7, wherein the (C₁-C₆)-alcohols are chosen from methanol, ethanol, propanol, isopropanol, butanol, pentanol, and hexanol.

9. A composition as claimed in claim 1, wherein the at least one physiologically tolerated film-forming agent comprises at least one naturally occurring substance chosen from alginic acid, alginates, collagen, collagen derivatives, hydrolyzed wheat proteins, carrageenan, cellulose, cellulose derivatives, chitosan, chitosan derivatives, keratin hydrolysates, protein hydrolysates, gelatin; guar gum, guar gum derivatives, hydrolyzed elastin, hydrolyzed milk proteins, hydrolyzed silk proteins, hydrolyzed soya proteins, hydrolyzed oat proteins, copolymers of hydroxyethylcellulose, dimethyldiallylammonium chloride, hyaluronic acid, hyaluronates, tragacanth, and xanthan.

10. A composition as claimed in claim 1, wherein the at least one physiologically tolerated film-forming agent comprises at least one synthetic substance chosen from acrylate/acrylamide copolymers, acrylate copolymer, acrylate/octylacrylamide copolymers, acrylic acid ester copolymers, methacrylic acid copolymers, adipic acid/dimethyl-aminohydroxypropyldiethylenetriamine copolymers, methacrylic acid/methacrylic acid ester copolymers neutralized with 2-amino-2-methylpropanol, polyacrylic acid crosslinked with pentaerythritol ethers or sugar allyl ethers, polysiloxane/polyalkyl polyether copolymers, polysiloxanes, ethylene/acrylic acid ester copolymers, ethylene/vinyl acetate copolymers, methacryloylethylbetaine/methacrylic acid copolymers, octylacrylamide/acrylic acid ester/butylaminoethylmethacrylic acid copolymers, quaternized polyvinylpyrrolidone-dimethylaminoethylmethacrylic acid esters, polyvinylpyrrolidone/imidazolinium methochloride copolymers, sodium acrylate/dimethyldiallylammonium chloride copolymers, dimethyldiallylammonium chloride/sodium acrylate/acrylamide terpolymers, poly(dimethylsiloxane-copolyol-phospho-panthenoate), poly(methyl vinyl ether-maleic anhydride), poly(methyl vinyl ether-maleic acid monoalkyl ester), poly(vinylpyrrolidone), terpolymers based on pyrrolidone and acrylic acid compounds, poly(vinylpyrrolidone-dimethylaminoethylmethacrylic acid), polyvinyl-pyrrolidonefeicosene copolymers, polyvinylpyrrolidone/methacrylic acid ester/methacrylic acid terpolymers, polyvinylpyrrolidone/hexadecene copolymers, polyvinylpyrrolidone/polycarbamyl polyglycol ester, polyvinylpyrrolidone/vinyl acetate copolymers, vinylimidazolium methochloride/vinylpyrrolidone copolymers, acrylic acid/acrylic acid ester copolymers and terpolymers of vinylpyrrolidone, vinyl acetate, and vinyl propionate.

11. A composition as claimed in claim 1, further comprising at least one additive chosen from circulation-promoting compounds, angiotensin converting enzyme inhibitors, methylxanthine compounds, sodium channel openers, and hair growth-promoting compounds.

12. A composition as claimed in claim 11, wherein at least one circulation-promoting compound is chosen from dihydralazine, diisopropylamine, diazoxide, and calcium antagonists.

13. A composition as claimed in claim 12, wherein at least one calcium antagonist is chosen from nifedipine, nicardipine, verapamil, diltiazem, nisoldipine, nitrendipine, nivaldipine, isradipine, felodipine, nimodipine, gallopamil, fendiline, flunarizine, amlodipine, diperdipine, fluspirilene, primozide, fantofarone, nicergoline, cyclandelate, and 6-amino-4-piperidino-1,2-dihydro-1-hydroxy-2-iminopyrimidine.

14. A composition as claimed in claim 11, wherein at least one angiotensin converting enzyme inhibitor is chosen from quinapril, lisinopril, benzazepril, captopril, ramipril, fosinopril, cifazapril, and trandolapril.

15. A composition as claimed in claim 11, wherein at least one methylxanthine compound is chosen from pentoxifyllin, propentofyllin, and torbafyllin.

16. A composition as claimed in claim 11, wherein at least one sodium channel opener is chosen from 1-cyano-2-(1,1-dimethyl-propyl)-3-(3-pyridyl)guanidine and 5-alpha-reductase inhibitors.

17. A composition as claimed in claim 16, wherein at least one 5-alpha-reductase inhibitor is N-tert-butyl-3-oxo-4aza-5α-androst-1-ene-17β-carboxamide.

18. A composition as claimed in claim 11, wherein at least one hair growth-promoting compound is chosen from inner salts of 2,4-diamino-6-alkoxy-3-sulfoxypyrimidine hydroxide having from 1 to 6 carbon atoms in the alkoxy radical, pyridine 1-oxide compounds, and 2,6-diamino-1,3,5-triazine compounds.

19. A composition as claimed in claim 18, wherein at least one hair growth-promoting compound is an inner salt of 2,4-diamino-6-butoxy-3-sulfoxypyrimidine hydroxide.

20. A composition as claimed in claim 18, wherein at least one pyridine 1-oxide compound is 2,6-diamino-4-piperidinopyridine.

21. A composition as claimed in claim 18, wherein at least one 2,6-diamino-1,3,5-triazine compound is 2,6-diamino-4-butoxy-1,3,5-triazine 1-oxide.

22. A process for making a product for treatment of androgenic alopecia or hirsutism, comprising the step of forming said product by bringing together:
a) at least one physiologically tolerated film-forming agent;
b) at least one physiologically tolerated solvent;
c) at least one plasticizer; and
d) a compound of the formula I 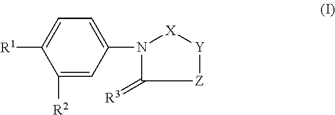
or a stereoisomeric form or a physiologically tolerated salt of any of the foregoing, in which:
R¹ is 1) —CN,
2) —NO₂,
3) a halogen, or
4) (C₁-C₄)-alkyl-C(O)—OH;
R² is 1) —CF₃,
2) a halogen, or
3) —CN;
R³ is 1) ═O,
2) ═S, or
3) ═NH;
X is 1) a radical of formula II 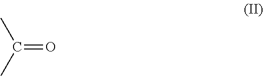
or
2) a radical of formula ill 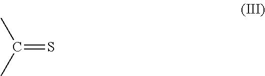
or X and Y together form a group of formula IV 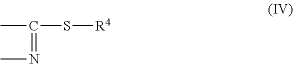
in which R⁴ is 1) hydrogen atom,
2) (C₁-C₆)-alkyl-,
3) (C₂-C₆)-alkenyl-, or
4) (C₁-C₆)-alkyl-,
wherein the alkyl is mono- to trisubstituted by
4.1 —OH,
4.2 halogens,
4.3 —O—(C₁-C₄)-alkyl,
4.4 —CN, or
4.5 —SH;
Y is 1) a radical of formula V 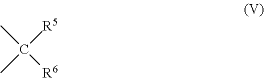
in which:
R⁵ is, independently of R⁶, a hydrogen atom or (C₁-C₄)-alkyl, wherein the alkyl is unsubstituted or mono- to tetrasubstituted by halogens, and
R⁶ is, independently of R⁵, (C₁-C₄)-alkyl, wherein the alkyl is unsubstituted or mono- to trisubstituted, by
a) halogens,
b) phenyl-(CH₂)ₘ—, wherein the phenyl is unsubstituted or mono- to trisubstituted, independently of one another, by —COOH, —CN, or —CF₃, and m is the integer zero, 1, 2, 3, 4, 5, or 6,
c) —COOH,
d) —CN, or
e) —CF₃, or
2) a radical of formula VI, 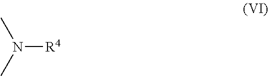
in which R⁴ is as defined above; and
Z is 1) —O— or
2) a radical of formula VII

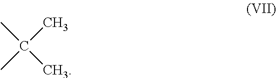

23. A process for making a product intended for treatment of seborrhea or acne, comprising the step of forming said product by bringing together:
a) at least one physiologically tolerated film-forming agent;
b) at least one physiologically tolerated solvent;
c) at least one plasticizer; and
d) a compound of the formula 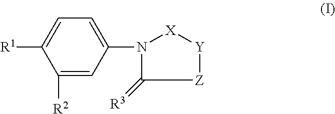
or a stereoisomeric form or a physiologically tolerated salt of any of the foregoing, in which:
R¹ is 1) —CN,
2) —NO₂,
3) a halogen, or
4) (C₁-C₄)-alkyl-C(O)—OH;
R² is 1) —CF₃,
2) a halogen, or
3) —CN;
R³ is 1) ═O,
2) ═S, or
3) ═NH;
X is 1) a radical of formula H 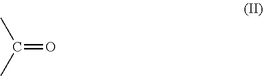
or
2) a radical of formula III 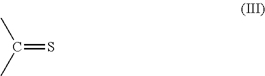
or X and Y together form a group of formula IV 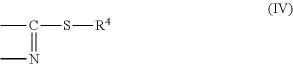
in which R⁴ is 1) hydrogen atom,
2) (C₁-C₆)-alkyl-,
3) (C₂-C₆)-alkenyl-, or
4) (C₁-C₆)-alkyl-,
wherein the alkyl is mono- to trisubstituted by
4.1 —OH,
4.2 halogens,
4.3 —O—(C₁-C₄)-alkyl,
4.4 —CN, or
4.5 —SH;
Y is 1) a radical of formula V 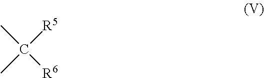
in which:
R⁵ is, independently of R⁶, a hydrogen atom or (C₁-C₄)-alkyl, wherein the alkyl is unsubstituted or mono- to tetrasubstituted by halogens, and
R⁶ is, independently of R⁵, (C₁-C₄)-alkyl, wherein the alkyl is unsubstituted or mono- to trisubstituted, by
a) halogens,
b) phenyl-(CH₂)ₘ—, wherein the phenyl is unsubstituted or mono- to trisubstituted, independently of one another, by —COOH, —CN, or —CF₃, and m is the integer zero, 1, 2, 3, 4, 5, or 6,
c) —COOH,
d) —CN, or
e) —CF₃, or
2) a radical of formula VI, 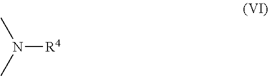
in which R⁴ is as defined above; and
Z is 1) —O— or
2) a radical of formula VII

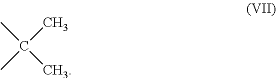

24. A process for treatment of androgenic alopecia or hirsutism, comprising the step of applying to a patient in need or desire thereof a composition comprising:
a) at least one physiologically tolerated film-forming agent;
b) at least one physiologically tolerated solvent;
c) at least one plasticizer; and
d) a compound of the formula I 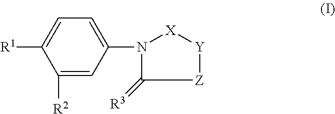
or a stereoisomeric form or a physiologically tolerated salt of any of the foregoing, in which:
R¹ is 1) —CN,
2) —NO₂,
3) a halogen, or
4) (C₁-C₄)-alkyl-C(O)—OH;
R² is 1) —CF₃,
2) a halogen, or
3) —CN;
R³ is 1) ═0,
2) ═S, or
3) ═NH;
X is 1) a radical of formula II 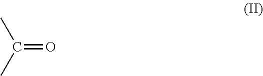
or
2) a radical of formula III 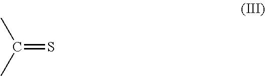
or X and Y together form a group of formula IV 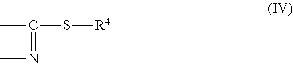
in which R⁴ is 1) hydrogen atom,
2) (C₁-C₆)-alkyl-,
3) (C₂-C₆)-alkenyl-, or
4) (C₁-C₆)-alkyl-,
wherein the alkyl is mono- to trisubstituted by
4.1 —OH,
4.2 halogens,
4.3 —O—(C₁-C₄)-alkyl,
4.4 —CN, or
4.5 —SH;
Y is 1) a radical of formula V 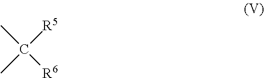
in which:
R⁵ independently of R⁶, a hydrogen atom or (C₁-C₄)-alkyl, wherein the alkyl is unsubstituted or mono- to tetrasubstituted by halogens, and
R⁶ is, independently of R⁵, (C₁-C₄)-alkyl, wherein the alkyl is unsubstituted or mono- to trisubstituted, by
a) halogens,
b) phenyl-(CH₂)ₘ—, wherein the phenyl is unsubstituted or mono- to trisubstituted, independently of one another, by —COOH, —CN, or —CF₃, and m is the integer zero, 1, 2, 3, 4, 5, or 6,
c) —COOH,
d) —CN, or
e) —CF₃, or
2) a radical of formula VI, 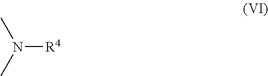
in which R⁴ is as defined above; and
Z is 1) —O— or
2) a radical of formula VII

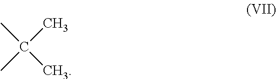

25. The process as claimed in claim 24, wherein said composition is applied to keratin fibers.

26. The process according to claim 25, wherein said fibers are human fibers.

27. The process according to claim 25, wherein said fibers are human hair.

28. A process for treatment of seborrhea or acne, comprising the step of applying to a patient in need or desire thereof a composition comprising:
a) at least one physiologically tolerated film-forming agent;
b) at least one physiologically tolerated solvent;
c) at least one plasticizer, and
d) a compound of the formula I 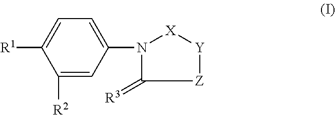
or a stereoisomeric form or a physiologically tolerated salt of any of the foregoing, in which:
R¹ is 1) —CN,
2) —NO₂,
3) a halogen, or
4) (C₁-C₄)-alkyl-C(O)—OH;
R² is 1) —CF₃,
2) a halogen, or
3) —CN;
R³ is 1) ═O,
2) ═S, or
3) ═NH;
X is 1) a radical of formula II 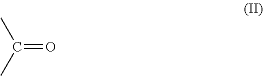
or
2) a radical of formula III 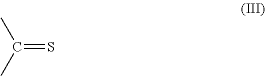
or X and Y together form a group of formula IV 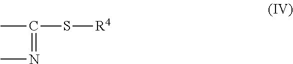
in which R⁴ is 1) hydrogen atom,
2) (C₁-C₆)-alkyl-,
3) (C₂-C₆)-alkenyl-, or
4) (C₁-C₆)-alkyl-,
wherein the alkyl is mono- to trisubstituted by
4.1 —OH,
4.2 halogens,
4.3 —O—(C₁-C₄)-alkyl,
4.4 —CN, or
4.5 —SH;
Y is 1) a radical of formula V 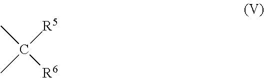
in which:
R⁵ is, independently of R₆, a hydrogen atom or (C₁-C₄)-alkyl, wherein the alkyl is unsubstituted or mono- to tetrasubstituted by halogens, and
R⁶ is, independently of R⁵, (C₁-C₄)-alkyl, wherein the alkyl is unsubstituted or mono- to trisubstituted, by
a) halogens,
b) phenyl-(CH₂)ₘ—, wherein the phenyl is unsubstituted or mono- to trisubstituted, independently of one another, by —COOH, —CN, or —CF₃, and m is the integer zero, 1, 2, 3, 4, 5, or 6,
c) —COOH,
d) —CN, or
e) —CF₃, or
2) a radical of formula VI, 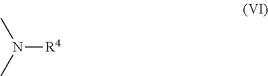
in which R⁴ is as defined above; and
Z is 1) —O— or
2) a radical of formula VII

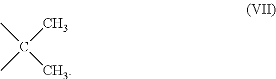

29. A cosmetic composition comprising:
a) at least one physiologically tolerated film-forming agent;
b) at least one physiologically tolerated solvent;
c) at least one plasticizer, and
d) a compound of the formula I 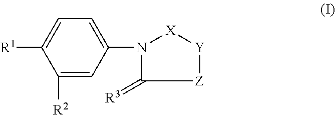
or a stereoisomeric form or a physiologically tolerated salt of any of the foregoing, in which:
R¹ is 1) —CN,
2) —NO₂,
3) a halogen, or
4) (C₁-C₄)-alkyl-C(O)—OH;
R² is 1) —CF₃,
2) a halogen, or
3) —CN;
R³ is 1) ═O,
2) ═S, or
3) ═NH;
X is 1) a radical of formula II 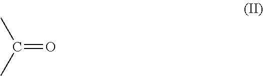
or
2) a radical of formula III 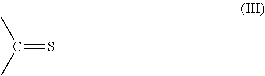
or X and Y together form a group of formula IV 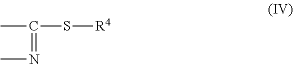
in which R⁴ is 1) hydrogen atom,
2) (C₁-C₆)-alkyl-,
3) (C₂-C₆)-alkenyl-, or
4) (C₁-C₆)-alkyl-,
wherein the alkyl is mono- to trisubstituted by
4.1 —OH,
4.2 halogens,
4.3 —O—(C₁-C₄)-alkyl,
4.4 —CN, or
4.5 —SH;
Y is 1) a radical of formula V 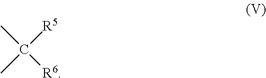
in which:
R⁵ is, independently of R⁶, a hydrogen atom or (C₁-C₄)-alkyl, wherein the alkyl is unsubstituted or mono- to tetrasubstituted by halogens, and
R⁶ is, independently of R⁵, (C₁-C₄)-alkyl, wherein the alkyl is unsubstituted or mono- to trisubstituted, by
a) halogens,
b) phenyl-(CH₂)ₘ—, wherein the phenyl is unsubstituted or mono- to trisubstituted, independently of one another, by —COOH, —CN, or —CF₃, and n1 is the integer zero, 1, 2, 3, 4, 5, or 6,
c) —COOH,
d) —CN, or
e) —CF₃, or
2) a radical of formula VI, 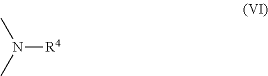
in which R⁴ is as defined above; and
Z is 1) —O— or
2) a radical of formula VII [chemical structure]
